(19) United States
(12) Patent Application Publication
(10) Pub. No.: US 2008/0033201 A1
(43) Pub. Date: Feb. 7, 2008

(54) Polyol Ester for Transformers

(76) Inventors: Matthias Hof, Duisburg (DE); Horst Baehr, Langenfeld (DE); Marc Moucha, Wuppertal (DE); Nigel Cooban, Merseyside (GB); Andreas Willing, Erkrath (DE)

Correspondence Address: COGNIS CORPORATION;PATENT DEPARTMENT, 300 BROOKSIDE AVENUE, AMBLER, PA, 19002, US

(21) Appl. No.: 11/569,485

(22) PCT Filed: May 18, 2005
(86) PCT No.: PCT/EP05/05375

§ 371 Date: Jul. 25, 2007

(30) Foreign Application Priority Data
May 27, 2004 (DE) 10 2004 025 939.9

(51) Int. Cl. C07C 69/34

(52) U.S. Cl. 560/201

(57) ABSTRACT

A dielectric insulating fluid composition for a transformer including a trimethylol propane ester corresponding to general formula I:

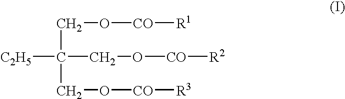

where R¹, R², and R³ are the same or different and each represents a linear or branched alkyl group containing 5 to 11 carbon atoms, where an ester having a viscosity of <23 mm²/s at 40° C. and a fire point of >300° C. is provided.

### RELATED APPLICATIONS

This application is a national phase filing under 35 U.S.C. § 371 of International Application No. PCT/EP2005/005375, filed on May 18, 2005, which designated the United States of America and which hereby claims priority from German Application DE 10 2004025939.9, filed on May 27, 2004, the entire disclosures of which are hereby incorporated herein by reference.

### FIELD OF THE INVENTION

This invention relates generally to transformer fluids, and more particularly to transformer fluids based on synthetic polyol esters and to their use as dielectric insulating fluids for transformers.

### BACKGROUND INFORMATION

High voltages are transported in transformers. Adequate electrical insulation is essential for safe operation. In addition, the heat generated in the transmission of electrical energy has to be dissipated. It is known that certain fluids have insulating and heat-dissipating properties. One of the problems involved in using these fluids is that the characteristics they are expected to show decrease with time and stress through high voltages. Mineral oils or silicone liquid are normally used. However, such fluids have poor biodegradability and are thus a danger to humans and the environment.

Accordingly, there is a growing demand for biodegradable oils for use as insulating fluids in transformers. Hitherto, this demand has been covered by the use of sunflower oil, rapeseed oil or soybean oil. Unfortunately, these oils do not have all the necessary properties in regard to oxidation stability.

Besides the oxidation stability already mentioned and thermal properties, such as flash point and fire point, the necessary properties also include pour point, a low acid value, dielectric stability and minimal sludge formation in the IEC stability test. In addition, good corrosion properties, seal compatibility and, above all, a stable viscosity are essential.

The oils used have to comply with IEC Standard 61099 “Specifications for unused synthetic organic esters for electricity purposes”. The flash point and fire point are also important characteristics.

The major advantage of mineral oils or silicone compounds is their low viscosity. The disadvantage of their poor bioldegradability and the absence or inadequacy of fire protection do not justify their use solely by virtue of the advantageous property.

EP 0292025 describes electrical appliances which are filled with a fire-retarding oil. The oil consists of an ester of a polyol with a flash point above 300° C. Polyol esters with a viscosity above the required range are described. In addition, no information is provided as to whether the polyols with the acid components used show good biodegradability.

Accordingly, the problem addressed by the present invention was to provide compounds as an alternative dielectric insulating liquid for transformers which would comply with IEC Standard 61099, would show very good biodegradability and would have low viscosities and high viscosity and oxidation stability with flash and fire points corresponding to the standard. In the most favorable case, the dielectric insulating fluid would be based on renewable raw materials and would be simple and inexpensive to produce.

### SUMMARY OF THE INVENTION

Briefly described, according to an aspect of the invention, a dielectric insulating fluid composition for a transformer includes a trimethylol propane ester corresponding to general formula I:

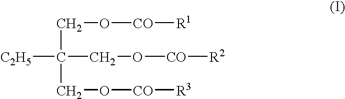

wherein R¹, R², and R³ are the same or different and each represents a linear or branched alkyl group containing 5 to 11 carbon atoms, wherein the ester has a viscosity of <23 mm²/s at 40° C. and a fire point of >300° C.

### DETAILED DESCRIPTION OF THE INVENTION

It has been found that trimethylol propane esters with saturated fatty acids as acid components satisfy the stated requirements in excellent fashion.

Accordingly, the present invention relates to compositions containing trimethylol propane esters (TMP esters) corresponding to general formula I:

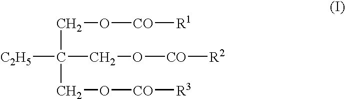

where R¹, R² and R³ may be the same or different and represent a linear or branched alkyl group containing 5 to 11 carbon atoms, the ester having a viscosity of <23 mm²/s at 40° C. and a fire point of >300° C. These esters are eminently suitable as dielectric insulating fluids for transformers.

The low viscosity which, in a preferred embodiment, is 20 mm²/s at 40° C. or lower leads to an improved heat-dissipating effect and, hence, to a longer useful life of the transformers. The low viscosity can be achieved by selected acid components for the esterification. The substituents R¹, R² and R³ in formula (I) consist of linear or branched C₅₋₁₁ alkyl groups. It is preferred to use substituents with linear or branched C₇₋₉ alkyl groups. The substituents have to be saturated to achieve the required oxidation stability. All the substituents in a polyol ester may be the same, only two may be the same or all may be different. Preference is attributed to a distribution of C₇₋₉ substituents which are formed in the esterification of trimethylol propane with an acid mixture of C₈₋₁₀ fatty acids, the fire point having to be above 300° C. and the viscosity reaching the described preferred ranges of <23 mm²/s at 40° C. The larger the number of C atoms, the higher the fire point, but the higher also the viscosity. Since these values run counter to one another, there is an optimum C chain distribution of the substituents R¹, R² and R³ for each pair of values.

This class of trimethylol propane esters comply with IEC 61099 and are classified as non-hazardous to water by the Deutsches Bundesamt für Umwelt (UBA, Berlin). Accordingly, their biodegradability is well above the standard of Water Hazard Class 1 and reaches a level of >60% BOD/COD or CO₂ evolution or >70% DOC removal after 28 days. This corresponds to the limits for ready biodegradability (as determined, for example, by OECD Method 301).

The compositions according to the invention have good thermal properties and excellent dielectric properties. In order further to improve the properties of the insulating fluid, antioxidants and/or metal deactivators may be, and preferably are, used. In another embodiment, the composition according to the invention additionally contains—based on the ester—0.005 to 1.0% by weight of an antioxidant and/or 0.01 to 2.0% by weight of a metal deactivator. The preferred quantity of antioxidant is between 0.01 and 0.5% by weight and, more particularly, is 0.1% by weight, based on the ester. The preferred quantity of metal deactivator is between 0.1 and 1.0% by weight and, more particularly, is 0.1% by weight, based on the ester.

The antioxidant is preferably selected from the group consisting of bis-hydroxytoluene, hydroquinone, 4-tert.butyl catechol, naphthol, phenyl naphthylamines, diphenylamines, phenylic thioethers, tocopherols and mixtures of the substances mentioned.

The metal deactivator is preferably an organic hetero compound selected from the group consisting of triazoles, tolyltriazoles, dimercaptothiadiazolines and mixtures of the substances mentioned.

The present invention also relates to the use of the composition according to the invention containing trimethylol propane esters (TMP esters) corresponding to general formula I:

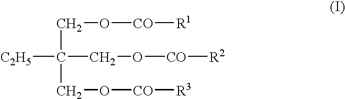

where R¹, R² and R³ may be the same or different and represent a linear or branched alkyl group containing 5 to 11 carbon atoms, the ester having a viscosity of <23 mm²/s at 40° C. and a fire point of >300° C., as dielectric insulating fluids for transformers.

The transformers in question are power transformers, distribution transformers, mast transformers, load stepping switches or change-over switches.

### EXAMPLES

### Production Example Ester T1

250 g trimethylol propane are mixed under nitrogen with 850 g fatty acid. After addition of 0.05 g tin(II) oxalate, the mixture is heated with vigorous stirring to 230° C. The reaction begins at 160° C. with intensive elimination of water. After reaching the reaction temperature of 230° C., the reaction is continued to an acid value of 20. Vacuum is then applied and the reaction completed at the same temperature. The reaction mixture is then cooled and the catalayst is neutralized by addition of 50% NaOH solution. The reaction mixture is dried and filtered through Celatom.

In a second step, 0.1% antioxidant and 0.1% metal deactivator are added to ester T1, an oxidation test is carried out to IEC 61099 and the characteristics are determined. The characteristics of a fully additive-charged ester, including the dielectric properties to IEC 61099, are set out by way of Example below:

| Physical properties | Unit | IEC 61099 | Ester T1 |
|---|---|---|---|
| Color | HU | <200 | 122 |
| Appearance | | Clear | Clear |
| Density at 20° C. | g/cm³ | <1 | 0.9451 |
| Kinematic viscosity | | | |
| at 40° C. | mm²/s | <35 | 19.3 |
| at −20° C. | mm²/s | <3000 | 468 |
| Flash point | ° C. | >250 | 273 |
| Fire point | ° C. | >300 | 313 |
| Crystal formation | | None | None |
| Pour point | ° C. | <−45° C. | −49 |

| Chemical Properties | Unit | IEC 61099 | Ester T1 |
|---|---|---|---|
| Water content | mg/kg | <200 | 100 |
| Neutralization value | mg KOH/g | <0.03 | 0.06 |
| Peroxide value | | | 0.2 |
| Oxidation stability | | | |
| Total acid content | mg KOH/g | <0.3 | 0.01 |
| Total sludge content | % by wt. | <0.01 | — |

| Electrical properties | Unit | IEG 61099 | Ester T1 |
|---|---|---|---|
| Breakdown voltage | kV | >45 | 75 |
| Dielectric loss factor | | <0.03 | 0.025 |
| at 90° C./50 Hz | | | |
| Direct-current resistance at 90° C. | Gohmm | >2 | 8.3 |

What is claimed is:

1. A dielectric insulating fluid composition for a transformer, comprising a trimethylol propane ester corresponding to general formula I: 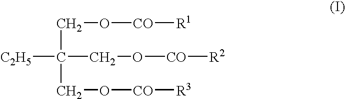
wherein R¹, R², and R³ are the same or different and each represents a linear or branched alkyl group containing 5 to 11 carbon atoms, wherein the ester has a viscosity of <23 mm²/s at 40° C. and a fire point of >300° C.

2. The composition according to claim 1, wherein R¹, R², and R³ are the same or different and each represents a linear or branched alkyl group containing 7 to 9 carbon atoms.

3. The composition according to claim 1, wherein the ester has a viscosity of 20 mm²/s at 40° C. or lower.

4. The composition according to claim 1, further comprising, based on the ester, 0.005% to 1.0% by weight of an antioxidant and/or 0.01% to 2.0% by weight of a metal deactivator.

5. The composition according to claim 4, wherein the antioxidant is selected from the group consisting of bis-hydroxytoluene, hydroquinone, 4-tert-butyl catechol, naphthol, phenyl naphthylamines, diphenylamines, phenylic thioethers, tocopherols, and mixtures thereof.

6. The composition according to claim 4, wherein the metal deactivator is an organic hetero compound selected from the group consisting of triazoles, tolyl triazoles, dimercaptothiadiazoles, and mixtures thereof.

7. The composition according to claim 1, incorporated into a transformer as a dielectric insulating fluid.

8. The composition according to claim 7, wherein the transformer is selected from the group consisting of power transformers, distribution transformers, mast transformers, load stepping switches, and change-over switches.
